(19) United States
(12) Patent Application Publication
(10) Pub. No.: US 2010/0274089 A1
(43) Pub. Date: Oct. 28, 2010

(54) LAPAROSCOPE AND SETTING METHOD THEREOF

(76) Inventors: Seung Wook Choi, Gyeonggi-do (KR); Dong Myung Min, Gyeonggi-do (KR)

Correspondence Address: BIRCH STEWART KOLASCH & BIRCH, PO BOX 747, FALLS CHURCH, VA, 22040-0747, US

(21) Appl. No.: 12/747,494

(22) PCT Filed: Oct. 1, 2009
(86) PCT No.: PCT/KR2009/005629

§ 371 Date: Jun. 10, 2010

(30) Foreign Application Priority Data
Oct. 24, 2008 (KR) 10-2008-0104522
Aug. 31, 2009 (KR) 10-2009-0080933

(51) Int. Cl. A61B 1/06

(52) U.S. Cl. 600/166

(57) ABSTRACT

A laparoscope includes: a housing extending a particular length; a pair of lenses set in both end portions of the housing along a lengthwise direction; a pair of first reflectors mounted within the housing adjacent to the pair of lenses to reflect light from the pair of lenses towards a particular position; a second reflector mounted within the housing that receives the light reflected from the pair of first reflectors and reflects the light in a particular direction; and an optical passage coupled to the housing that receives the light reflected from the second reflector and transmits the light to a particular position. Since a single-lens laparoscope may be connected to the housing where a pair of lenses are set with a gap in-between, the diameter of the laparoscope can be reduced, and an image can be obtained that has a brightness comparable to that obtained by a single-lens laparoscope.

### CROSS-REFERENCE TO RELATED APPLICATIONS

This application claims foreign priority benefits under 35 U.S.C. sctn. 119(a)-(d) to PCT/KR2009/005629, filed Oct. 1, 2009, which is hereby incorporated by reference in its entirety.

### BACKGROUND

The present invention relates to a laparoscope and to a method of setting the laparoscope.

In the field of medicine, surgery refers to a procedure in which a medical device is used to make a cut or an incision in or otherwise manipulate a patient's skin, mucosa, or other tissue, to treat a pathological condition. One type of surgery, a laparotomy, is a surgical procedure in which the skin of the abdomen is cut open and an internal organ, etc., is treated, reconstructed, or excised.

When conducting a laparotomy, an incision is made in the skin and a particular amount of space is formed between the skin and the tissue, with the surgical operation performed within this space. As this may increase scars and prolong the healing period, laparoscopic surgery has recently been proposed as an alternative.

In laparoscopic surgery, a small incision is made in the surgical site of the patient, through which a laparoscope is inserted, so that the surgery may be conducted while observing the surgical site within the abdominal cavity. Laparoscopic surgery is widely used in various fields of medicine, including internal medicine, surgery, urology, gynecology, and obstetrics. The laparoscope is an apparatus used for the imaging diagnosis of an internal organ and typically involves an apparatus mounted with a miniature camera that is inserted in the body, so that the image information retrieved by the miniature camera may be observed through an external monitor.

The conventional laparoscope can be divided into the single-lens type, which includes one lens set in an end portion of a cylindrical tube, and the stereo type, which includes a pair of lenses. The single-lens type provides a relatively brighter image, but is able to provide only a planar image that does not render a 3-dimensional look, whereas the stereo type is able to provide a 3-dimensional image, but provides an image that is not as bright. In addition, even when a stereo laparoscope is used, the 3-dimensional effect of the image may not be significant, as the pair of lenses may be set within a range of about 12 mm, the diameter of a typical laparoscope.

In “robot surgery,” which is performed using a laparoscope and a surgical robot inserted in the patient's body, an incision may be made in the patient's skin, and a trocar may be inserted that serves as a port through which the laparoscope and surgical instruments are inserted. Since the laparoscope may require a minimum cross section for setting a pair of lenses as described above, it is unsuitable to indiscriminately reduce the size of the laparoscope, unlike other surgical equipment. Consequently, in the case of robot surgery, the trocar used for inserting the laparoscope currently has the largest diameter.

In conventional laparoscopic surgery, a gas such as CO2 may be injected into the portion photographed with the laparoscope, in order to obtain a space required for photography. The injected gas is not harmless to the human body, and there is also a risk of a medical accident if the gas pressure is not properly regulated.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the present invention is to provide a laparoscope and a method of setting the laparoscope, which can be used to obtain an image having a significantly 3-dimensional effect and having a brightness level comparable to that of the single-lens type, even with a reduced diameter of the laparoscope, which do not require the injection of a gas such as CO2 to obtain a space for laparoscopic photography, and which do not require making an incision in the skin for inserting the laparoscope.

One aspect of the present invention provides a laparoscope that includes: a housing, which extends a particular length; a pair of lenses, which are set in both end portions of the housing along a lengthwise direction; a pair of first reflectors, which are mounted within the housing adjacent to the pair of lenses to reflect light from the pair of lenses towards a particular position; a second reflector, which is mounted within the housing, and which receives the light reflected from the pair of first reflectors and reflects the light in a particular direction; and an optical passage, which is coupled to the housing, and which receives the light reflected from the second reflector and transmits the light to a particular position.

The laparoscope can further include a pair of first polarizing filters that polarize in different directions the light received through the pair of lenses, respectively, while the laparoscope can include a pair of second reflectors that correspond respectively to the pair of first reflectors, and one of the pair of second reflectors can include a two-way mirror to reflect light reflected from the corresponding first reflector and transmit light reflected by the other of the pair of second reflectors. In this case, the laparoscope can further include an optical splitter, for receiving the light transmitted by the optical passage and separating the light into two channels, and a pair of second polarizing filters, for respectively polarizing the separated light in same directions as the pair of first polarizing filters.

The optical passage can be coupled to the housing along the same lengthwise direction as that of the housing, and an auxiliary lens can be set in an end portion along the lengthwise direction of the housing, for checking a forward image when the laparoscope is inserted into the body. A shutter can be set in the housing to selectively block the pair of lenses and the auxiliary lens.

Another aspect of the present invention provides a laparoscope that includes: a housing, which extends a particular length; a pair of lenses, which are set in both end portions of the housing along a lengthwise direction; an image sensor, which is mounted within the housing, and which receives light through each of the pair of lenses, converts the light into electrical signals, and generates image information corresponding to the converted electric signals; and a transmitter, which transmits the image information to a receiver device.

The laparoscope can further include a support, which may be detachably coupled to the housing, and which may extend along a particular lengthwise direction, where the support can be coupled to the housing along the same lengthwise direction as that of the housing. An auxiliary lens can be set in an end portion along the lengthwise direction of the housing, for checking a forward image while the laparoscope is inserted into the body. A shutter can be set in the housing to selectively block the pair of lenses and the auxiliary lens.

A stopper can be included on the optical passage (or the support) to restrict the rotation of the housing when the housing is rotated to an orientation having a same lengthwise direction as that of the optical passage (or the support) or to an orientation orthogonal to the optical passage (or the support).

The transmitter can be implemented as a wired communication system, for example using electrical contacts, etc., or can include a wireless communication module for exchanging data by wireless communication.

The laparoscope can further include a magnet coupled to the housing, and the laparoscope, after being inserted into a body, can be secured to a particular position by a magnetic force applied from out of the body. Also, the laparoscope, after being inserted into a body, can be secured to a particular position by a hook that has one end coupled to the housing and the other end coupled to an abdominal wall of the patient.

A power source such as a battery, etc., can additionally be included for supplying power to the image sensor and the transmitter. The power source can also generate an induced current by electromagnetic induction from a relationship with a power supply device located outside the housing.

The housing can include a base unit and a module unit, where the module unit can be coupled to the base unit such that the module unit is retractable and protractible from the base unit, and the lenses and the image sensor can be held in the module unit. In this case, a pair of module units can be coupled respectively to both sides of the base unit, and a lens and an image sensor can be held in each of the pair of module units such that retracting or protracting the pair of module units from the base unit may decrease or increase the gap between the pair of lenses. Also, the module unit can be coupled to the base unit such that the module unit is rotatable about a particular axis, so that rotating the module unit may change the angle by which the lens views an object.

A distance sensor can be coupled to the housing that generates a sensing signal corresponding to a distance to the object, while the laparoscope can further include a computing unit, for receiving a signal from the distance sensor and calculating a convergence angle of the pair of module units with respect to the object, and a driving unit, for rotating the module units in accordance with the angle calculated by the computing unit. Alternatively, the laparoscope can include a computing unit that analyzes an image obtained from the image sensor, produces data corresponding to a focal length for the object, and calculates a convergence angle of the pair of module units from the produced data, and a driving unit that rotates the module units in accordance with the angle calculated by the computing unit.

In such cases, the driving unit can be coupled to the pair of module units, so that the pair of module units may be rotated in linkage, or a pair of driving units can be coupled respectively to the pair of module units so that the pair of module units may be rotated separately.

Yet another aspect of the present invention provides a stereo adapter for use in a laparoscope. The stereo adapter, which can be mounted on a laparoscope to obtain a stereo image, may include: an optical passage, in which the laparoscope is inserted; a housing, which is rotatably coupled to the optical passage, and which extends a particular length; a pair of lenses, which are set in both end portions of the housing along a lengthwise direction; a pair of first reflectors, which are mounted within the housing adjacent to the pair of lenses, and which reflect light from the pair of lenses towards a particular position; and a second reflector, which is mounted within the housing, and which is configured to receive the light reflected from the pair of first reflectors and reflect the light towards the laparoscope.

The second reflector can be made to undergo oscillatory rotation at a particular frequency, such that the second reflector may receive the light reflected from the pair of first reflectors and reflect the light respectively towards the laparoscope. In this case, the oscillatory rotation frequency of the second reflector can be 60 to 120 Hz.

The optical passage can be detachably coupled to the housing. This can be implemented by a magnet attached to one or more of the optical passage and the housing that couples the optical passage to the housing by magnetic force, or by an alignment protrusion formed on the end portion of the optical passage coupled to the housing and an indentation mating formed in the portion of the housing coupled to the optical passage that mates with the alignment protrusion.

The optical passage can be coupled to the housing along the same lengthwise direction as that of the housing. Alternatively, the housing can be hinge-coupled to the optical passage, such that the housing is rotatable between an orientation having a same lengthwise direction as that of the optical passage and an orientation orthogonal to the optical passage.

Yet another aspect of the present invention provides a stereo adapter for mounting on a laparoscope, which polarizes in different directions the light received through a pair of lenses respectively through a pair of first polarizing filters and transmits the polarized light to a single channel. The stereo adapter includes: an optical splitter, which receives the light transmitted by the laparoscope and separates the light into two channels; and a pair of second polarizing filters, which respectively polarize the separated light in same directions as the pair of first polarizing filters.

Additional aspects, features, and advantages, other than those described above, will be obvious from the claims and written description below.

According to an embodiment of the present invention, a single-lens laparoscope may be connected to a housing in which a pair of lenses are set with a particular gap in-between, so that the diameter of the laparoscope can be reduced, and an image can be obtained that has a brightness comparable to that obtained by a single-lens laparoscope. Since the pair of lenses can be separated as necessary to a distance similar to the distance between human eyes, an image can be obtained that provides a 3-dimensional effect similar to that observed by human eyes. Furthermore, by setting a lighting device, such as LED's, etc., onto the housing, the laparoscopic surgery can be performed with a much wider range of vision.

Also, the housing equipped with the lenses and a CCD can be inserted as a laparoscope module into the body, after which a space can be obtained by pulling on the laparoscope module. In this way, the space required for laparoscopic photography can be obtained without injecting a gas, such as CO2, etc. The laparoscope module can be inserted through an incision made for a different purpose, with only the needle protruded outside. Thus, since there is no need to make an incision in the skin for inserting the laparoscope, a safer form of “minimally invasive surgery” can be implemented.

### BRIEF DESCRIPTION OF THE DRAWINGS

FIG. 1 is a cross-sectional view of a laparoscope according to an embodiment of the present invention.

FIG. 2 through FIG. 4 are cross-sectional views of laparoscopes according to embodiments of the present invention.

FIG. 5 through FIG. 7 are cross-sectional views of laparoscopes according to embodiments of the present invention.

FIG. 8 is a plan view of a laparoscope according to an embodiment of the present invention.

FIG. 9 and FIG. 10 are plan views of laparoscopes according to embodiments of the present invention.

FIG. 11 through FIG. 13 are cross-sectional views of laparoscopes according to embodiments of the present invention.

FIG. 14 through FIG. 19 illustrate the operation of certain laparoscopes according to embodiments of the present invention.

FIG. 20 and FIG. 21 illustrate the use of certain laparoscopes according to embodiments of the present invention.

FIG. 22 and FIG. 23 are cross-sectional views of laparoscopes according to embodiments of the present invention.

FIG. 24 is a plan view of a laparoscope according to an embodiment of the present invention.

FIG. 25 is a magnified cross-sectional view of portion “S” in FIG. 8 and FIG. 24.

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the written description, certain detailed explanations of related art are omitted when it is deemed that they may unnecessarily obscure the essence of the present invention.

While such terms as “first” and “second,” etc., may be used to describe various components, such components must not be limited to the above terms. The above terms are used only to distinguish one component from another.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Certain embodiments of the present invention will be described below in detail with reference to the accompanying drawings. Those components that are the same or are in correspondence are rendered the same reference numeral regardless of the figure number, and redundant descriptions are omitted.

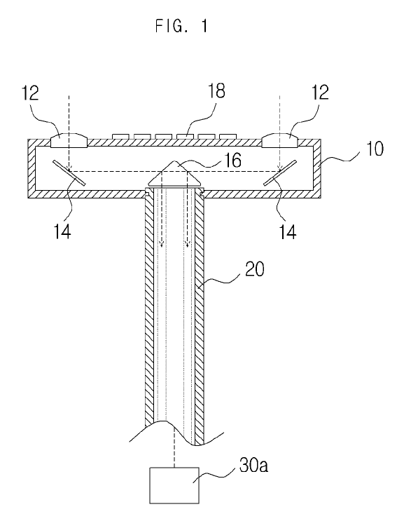

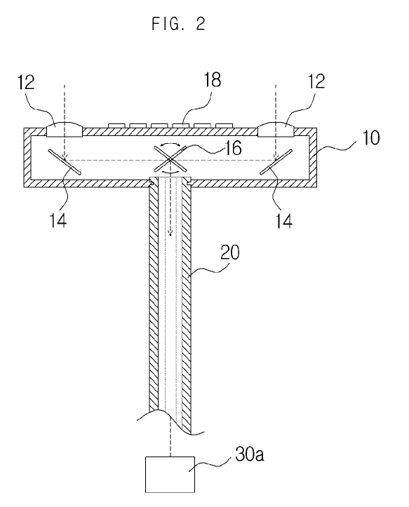

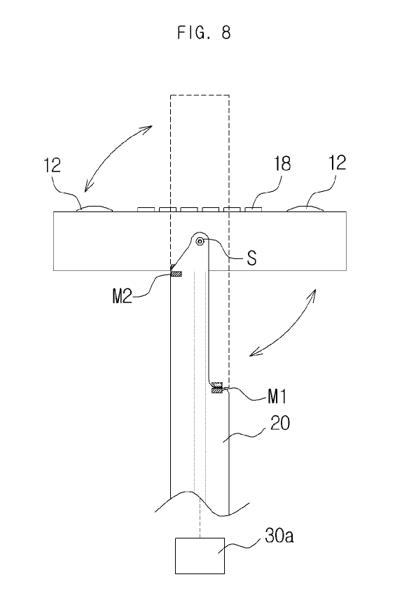

FIG. 1 is a cross-sectional view of a laparoscope according to an embodiment of the present invention, FIG. 2 through FIG. 4 are cross-sectional views of laparoscopes according to embodiments of the present invention, FIG. 5 through FIG. 7 are cross-sectional views of laparoscopes according to embodiments of the present invention, and FIG. 8 is a plan view of a laparoscope according to an embodiment of the present invention. Illustrated in FIG. 1 to FIG. 8 are a housing 10, lenses 12, first reflectors 14, a second reflector 16, a lighting part 18, an optical passage 20, and an optical receiver 30a.

This embodiment discloses a laparoscope which resolves the drawbacks of conventional stereo laparoscopes, namely, the limitation in reducing the diameter and the low brightness of the image, and which provides a superb 3-dimensional image comparable to that observed by human eyes.

In the present embodiment, a pair of lenses can be set with the distance in-between similar to the distance between the eyes of an actual person, so that the 3-dimensional effect may be greatly improved compared to that of the conventional stereo laparoscope.

That is, the structure of a laparoscope according to the present embodiment can include the pair of lenses 12 set in the housing 10 with a particular distance in-between, where the light from the lenses 12 may be reflected and then transmitted via the optical passage 20 to the optical receiver 30a. The optical passage 20 can be modified from an existing laparoscope such that the portion coupled to the housing 10 may be detachably joined to the housing 10.

If a mirror undergoing oscillatory rotation at a particular frequency is used to transmit the light from each of the pair of lenses 12 respectively to the optical passage 20, as will be described later in more detail, an existing single-lens laparoscope can be applied unaltered as the optical passage 20. As such, the diameter of the laparoscope, i.e. the diameter of the optical passage 20 can be greatly reduced, compared to that of the conventional stereo laparoscope.

The housing 10 may be shaped as a tube that extends along a particular length, such that the pair of lenses 12 may be arranged with a particular gap in-between. In the example shown in FIG. 1, the housing 10 is formed as a cylindrical pipe, and the pair of lenses 12 are set in both ends of the housing 10, so that the pair of lenses 12 are separated from each other by the length of the housing 10. Therefore, in a laparoscope according to the present embodiment, the gap between the pair of lenses 12 may be increased simply by increasing the length of the housing 10 correspondingly.

A reflector may be mounted within the housing 10 to transfer the light from the lenses 12 to the optical passage 20. That is, a pair of first reflectors 14 may be mounted adjacent to the pair of lenses 12, to reflect the light from the lenses 12 respectively to a second reflector 16, and the second reflector 16 may be mounted to receive the light from the pair of first reflectors 14 and reflect the light to the optical passage 20. Various optical systems, such as mirrors and prisms, etc., which are capable of altering an optical path, can be applied as the first reflectors 14 and second reflector 16.

FIG. 1 illustrates a mechanism in which mirrors are set as the first reflectors 14 and a prism is set as the second reflector 16 to transfer the light from the lenses 12 to the optical passage 20, but the present invention is not thus limited, and it is obvious that various other optical systems for transferring the light from the lenses 12 to the optical passage 20 can be used in addition to or in place of the first and second reflectors 14, 16.

In this way, the light from the lenses 12 may be transferred by the first and second reflectors 14, 16 to the optical passage 20, and may then be transmitted via the optical passage 20 to an optical receiver 30a coupled to the end of the optical passage 20. The optical receiver 30a may be equipped with a device for receiving and processing image information, such as a CCD (charge-coupled device), etc., so that the image photographed by the laparoscope may be displayed on a monitor.

As the light from the pair of lenses 12 are reflected by the first reflectors 14, respectively, and transferred to the second reflector 16, the second reflector 16 may receive a pair of image information sets. In order to reflect the pair of image information sets to the optical passage 20, the second reflector 16 can be formed in the shape of a prism, as in the example shown in FIG. 1. In this case, an existing stereo laparoscope can be used for the optical passage 20, so that the pair of image information sets reflected by the second reflector 16 may be transmitted simultaneously to the optical receiver 30a.

Alternatively, when using a single-lens laparoscope for the optical passage 20 as described above to reduce the diameter of the laparoscope, the second reflector 16 can be formed as a mirror that undergoes oscillatory rotation at a particular frequency, as in the example shown in FIG. 2. In other words, the pair of image information sets entering the second reflector 16 may be reflected alternately to the optical passage 20.

For example, if an image information set from one lens 12 is to be received at 30 fps (frames per second), the second reflector 16 can be oscillated at 60 Hz, so that the image information obtained from one of the lenses 12 may be transmitted at 30 frames per second to the optical receiver 30a. Likewise, if an image of 60 fps is desired, the second reflector 16 may be rotated at 120 Hz.

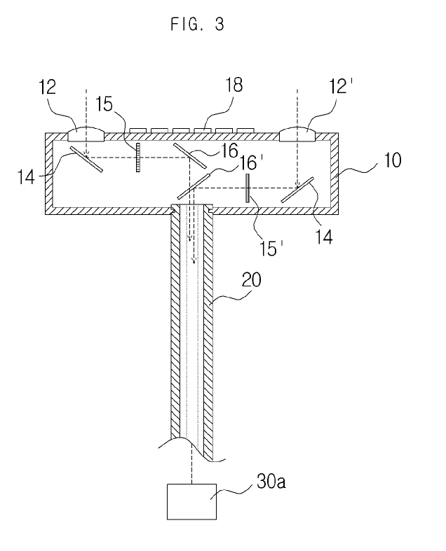

As an alternative to this type of structure in which the second reflector is oscillated, FIG. 3 illustrates a structure in which the images from the pair of lenses 12, 12′, i.e. the left-eye image and the right-eye image, can be obtained collectively through a single optical passage 20 by using polarizing filters 15, 15′ and a two-way mirror 16′.

That is, the light from the left lens 12 may be reflected on a first reflector 14, passed through a polarizing filter 15, and reflected by a second reflector 16 towards the optical passage 20, while the light from the right lens 12′ may be reflected on a first reflector 14, passed through a polarizing filter 15′, and reflected by a second reflector 16 towards the optical passage 20. If, for example, the left polarizing filter 15 is made to polarize light in a lateral direction and the right polarizing filter 15′ is made to polarize light in a longitudinal direction, the left-eye image and the right-eye image can both be reflected collectively towards the optical passage 20 and separated again as the left-eye and right-eye images.

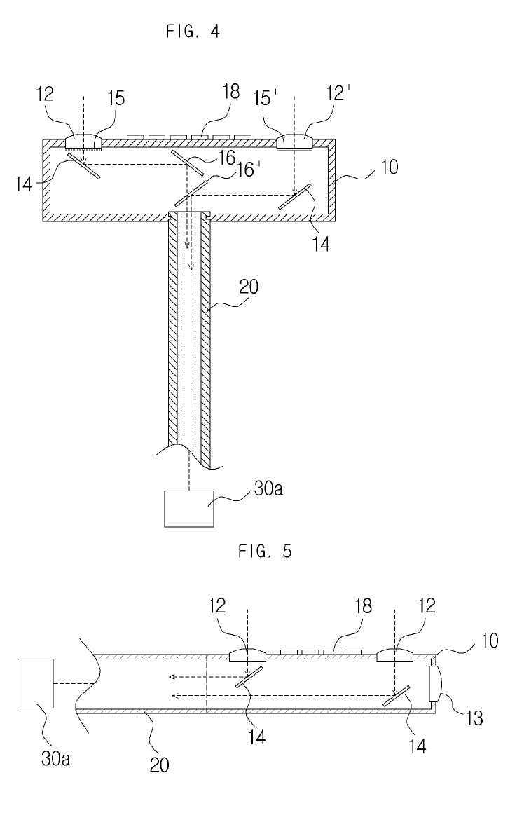

While FIG. 3 illustrates an example in which the polarizing filters 15, 15′ are positioned respectively between the first reflectors 14 and the second reflectors 16, 16′, the positions of the polarizing filters according to this embodiment are not thus limited, and it is obvious that the polarizing filters can be positioned elsewhere to polarize the light obtained through the respective lenses 12, 12′. For example, the polarizing filters can be positioned between the lenses 12, 12′ and the first reflectors 14, or as illustrated in FIG. 4, the polarizing filters 15, 15′ can be coupled to the pair of lenses 12, 12′, respectively.

Also, as illustrated in FIG. 3, one of the two second reflectors 16, 16′ can be a two-way mirror, so that the light reflected from one second reflector 16 may be transmitted through the other 16′. Thus, the two-way mirror may transmit the light from the left lens 12 but reflect the light from the right lens 12′, whereby all of the light from the pair of lenses 12, 12′ can be directed towards the optical passage 20.

As such, the left-eye image and the right-eye image may both pass through the optical passage 20 according to this embodiment, and as described above, the left-eye image can be polarized in a lateral direction, while the right-eye image can be polarized in a longitudinal direction.

In such cases where the left-eye and right-eye images polarized in different directions are obtained collectively through the optical passage 20, an optical splitter can be used on the side of the optical receiver 30a to separate the light into two channels, after which the light may be passed through a lateral and a longitudinal polarizing filter, respectively, so that the image polarized in a lateral direction may be obtained as a left-eye image, and the image polarized in a longitudinal direction may be obtained as a right-eye image.

Of course, the polarizing filters according to this embodiment do not necessarily have to polarize the light in a lateral and a longitudinal direction, and other filters can be used that make it possible to polarize light in different directions and separate the light back into images after the images are obtained collectively.

With this embodiment, it is not needed to rotate the second reflector at a particular frequency as in the example illustrated in FIG. 2, so that a structure can be implemented that is simpler in terms of electrical and mechanical composition.

This structure for transmitting the left-eye and right-eye images through a single channel and then dividing the images again does not necessarily have to be applied to a system integrated with a laparoscope, and can instead be implemented in the form of an adapter that can be mounted on the back end of the laparoscope.

That is, as illustrated in FIG. 10, a stereo adapter 110 can be manufactured separately that includes an optical splitter 112 and polarizing filters 115. The adapter 110 can be mounted on the back end of a laparoscope 1, and the single-channel image transmitted through the laparoscope 1 can be divided into two channels, so that the left-eye image and right-eye image may be obtained separately.

The stereo adapter 110 according to this embodiment can be mounted onto the back end of a laparoscope 1 that transmits the left-eye and right-eye images through a single channel, whereby a stereo image can be obtained simply by mounting the adapter 110 without having to modify the form or structure of the laparoscope.

Since the optical passage 20 according to the present embodiment may be inserted into the abdominal cavity independently of the housing 10 and may be coupled to the housing 10 afterwards, the optical passage 20 can be detachably coupled to the housing 10. When using an existing laparoscope for the optical passage 20 as described above, the end portion of the laparoscope can be modified to a structure that enables joining to the housing 10.

This can be achieved by forming an alignment protrusion (not shown) on an end portion of the optical passage 20 and forming a corresponding indentation (not shown) in the housing 10, so that the optical passage 20 and the housing 10 may be automatically aligned simply by mating the alignment protrusion with the indentation when coupling the optical passage 20 with the housing 10. Of course, it is also possible to form the alignment indentation in an end portion of the optical passage 20 and form a corresponding protrusion that mates with the alignment indentation on the housing 10, and it is obvious that various other mechanical structures may be utilized to align the optical passage 20 with the housing 10.

In addition, it is possible to attach a pair of magnets (not shown) respectively to opposing portions of the optical passage 20 and the housing 10, so that the optical passage 20 and the housing 10 may be attached and detached by magnetic force. The pair of magnets can be a pair of permanent magnets or electromagnets that applies an attractive force on each other, or a mixed set of a permanent magnet and an electromagnet. It is also possible to attach a magnet to one of the optical passage 20 and the housing 10 and attach a magnetic substance, such as a piece of metal, to the other.

As set forth above, the laparoscope according to the present embodiment is a system that uses a pair of lenses 12 to collect image information and uses an optical passage 20 and an optical receiver 30a to process the information into a binocular image. That is, through the shared use of the optical passage 20, as well as the CCD camera coupled to the end of the optical passage 20, a binocular image can be obtained using only a single optical receiver 30a.

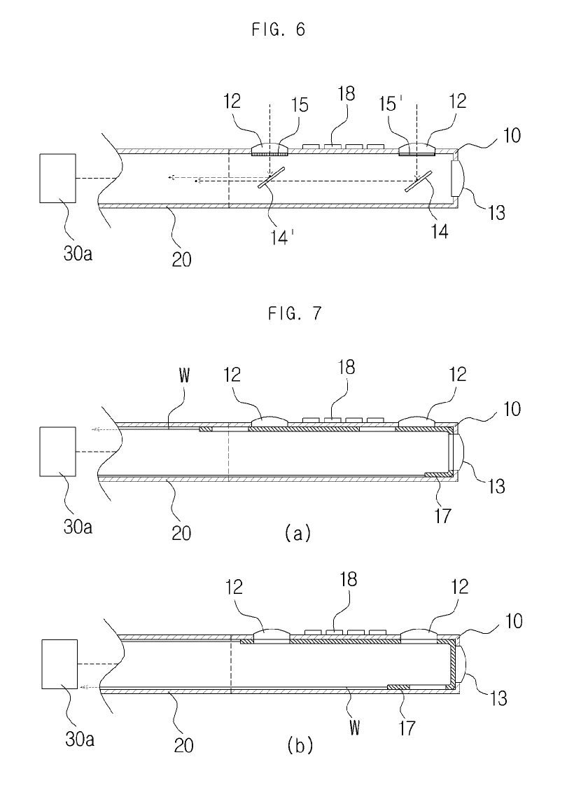

The optical passage 20 does not necessarily have to be coupled to the housing 10 orthogonally, and as illustrated in FIG. 5 and FIG. 6, the optical passage 20 can be coupled to the housing 10 such that the lengthwise direction of the optical passage 20 matches the lengthwise direction of the housing 10.

FIG. 5 illustrates a structure (a dual-channel structure) in which the light from the pair of lenses 12 is respectively reflected to the optical receiver 30a, and FIG. 6 illustrates a structure (a single-channel structure) in which the light from the pair of lenses 12, i.e. the left-eye image and the right-eye image, is obtained collectively by an arrangement including polarizing filters 15, 15′ and a two-way mirror 14′, similar to the example illustrated in FIG. 3.

As in the examples shown in FIG. 5 through FIG. 7, a separate auxiliary lens 13 can additionally be set in an end portion of the housing 10, in order to check the forward image during the process of inserting the laparoscope into the body

In this case, the auxiliary lens 13 can be used during the insertion of the laparoscope into the body, and after the laparoscope is inserted, the pair of lenses 12 can be used. In order to selectively use or block the lenses according to circumstances, a shutter 17 may additionally be mounted in the housing.

That is, a shutter 17, which may include apertures that correspond with the pair of lenses 12 and the auxiliary lens 13, can be installed in the housing 10, where the apertures corresponding with the respective lenses may be moved by pulling a wire (“W” in FIG. 7), etc., coupled to the end of the shutter 17. When only the auxiliary lens 13 is to be used, during the process of inserting the laparoscope, a wire W can be pulled such that the auxiliary lens 13 is aligned with an aperture but the pair of lenses 12 are blocked by the shutter 17, as in illustration (a) of FIG. 7. When only the pair of lenses 12 are to be used, after the laparoscope is inserted into the body, a wire W can be pulled such that the pair of lenses 12 are aligned with the apertures but the auxiliary lens 13 is blocked by the shutter 17, as in illustration (b) of FIG. 7.

Also, as in the embodiment illustrated in FIG. 5 and FIG. 6, the optical passage 20 and the housing 10 do not necessarily have to be formed as a detachable structure, and may be used as an integrated part.

As illustrated in FIG. 8, the housing 10 can be hinge-coupled to the optical passage 20, such that the housing 10 is able to rotate in relation to the optical passage 20. When the laparoscope according to the present embodiment is inserted into the body, the housing 10 can be positioned to have the same lengthwise direction as that of the optical passage 20, and after the laparoscope is inserted into the body, the housing 10 can be rotated such that the housing 10 is orthogonal to the optical passage 20, to continue with the laparoscopic photography.

In this case also, it is not necessary to join the housing 10 and the optical passage 20 after inserting the housing 10 and the optical passage 20 separately into the body. The housing 10 can be inserted into the body through the trocar while joined to the optical passage 20, and afterwards the housing 10 can be rotated for use.

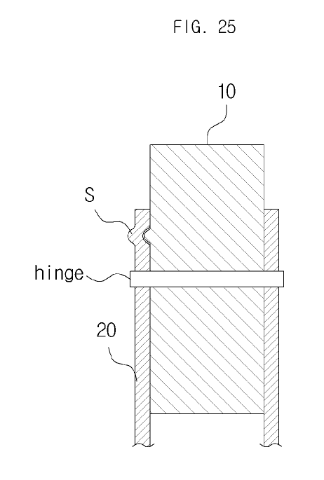

In order that the housing 10 may maintain its orientation after it has been rotated to have the same lengthwise direction as that of the optical passage 20, a stopper (“S” in FIG. 8) can be formed near the hinge as illustrated in FIG. 25, or a pair of magnets (“M1” in FIG. 8) can be set in the appropriate positions of the housing 10 and the optical passage 20 to serve as a stopper. It is also possible to form a stopper (“S” in FIG. 8, refer to FIG. 25) near the hinge or set a pair of magnets (“M2” in FIG. 8) in the appropriate positions of the housing 10 and the optical passage 20 to serve as a stopper, for the purpose of maintaining the orientation of the housing 10 after it has been rotated to be orthogonal to the optical passage 20.

The structure illustrated in FIG. 8 does not necessarily have to be applied to a laparoscope, and may be implemented as an adapter, which may be mounted on an existing laparoscope to obtain a stereo image.

In other words, an adapter 100 composed mainly of the optical passage 20 and a housing 10 hinge-coupled thereto can be manufactured, as illustrated in FIG. 9, where the adapter 100 may be mounted on an existing laparoscope 1, i.e. the laparoscope 1 may be inserted into the optical passage 20, so that the existing laparoscope 1 may be used as a stereo laparoscope that provides an excellent 3-dimensional effect. For example, if an existing laparoscope is assumed to have a diameter of 10 mm, the optical passage according to this embodiment can be designed to have a diameter of 12 mm, so that the existing laparoscope may be inserted into the optical passage.

If a stereo adapter 100 according to this embodiment is mounted on an existing laparoscope 1, the light from the pair of lenses 12 may be reflected by the first and second reflectors into the existing laparoscope 1, and as such, the existing laparoscope 1 can be used as a stereo laparoscope, without having to modify the form or structure, simply by mounting an adapter 100 according to the present embodiment.

In this case, the second reflector included in the adapter can be made to undergo oscillatory rotation at a particular frequency (e.g. 60 to 120 Hz) to receive the light reflected from the pair of first reflectors and reflect the light respectively towards the laparoscope, and the optical passage can be detachably joined to the housing by using magnets or an alignment protrusion and indentation, etc., similar to the case of the laparoscope described above.

The optical passage can be coupled to the housing along the same lengthwise direction as that of the housing, as in the examples described with reference to FIG. 5 through FIG. 7. Furthermore, similar to the example in FIG. 8, the housing 10 can be hinge-coupled to the optical passage 20 such that the housing 10 is able to rotate between an orientation having the same lengthwise direction as that of the optical passage 20 and an orientation orthogonal to the optical passage 20. Thus, when the adapter 100 mounted on an existing laparoscope 1 is inserted into the body, the housing 10 can be made to have the same lengthwise direction as the optical passage 20. Then, after the adapter 100 is inserted into the body, the housing 10 can be rotated to be orthogonal to the optical passage 20, and laparoscopic photography may be performed after pushing the laparoscope 1 in such that the lens of the laparoscope 1 is close to the second reflector.

It is also conceivable to mount the adapter 100 illustrated in FIG. 9 on the front end of a laparoscope while mounting the adapter 110 illustrated in FIG. 10 on the back end of the laparoscope. That is, a regular single-channel laparoscope 1 can be used for the optical passage, but with an adapter 100 for collecting the stereo images (the left-eye and right-eye images) mounted on the front end and an adapter 110 for dividing the collected image back into stereo images (the left-eye and right-eye images) mounted on the back end, so that the regular laparoscope may be used as a stereo laparoscope according to the present embodiment described above.

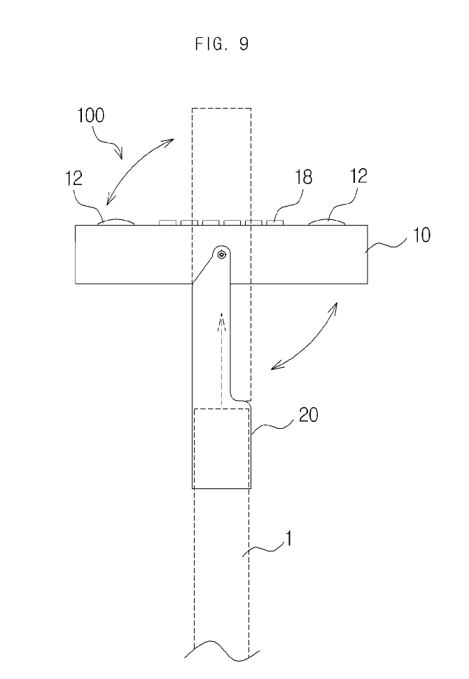

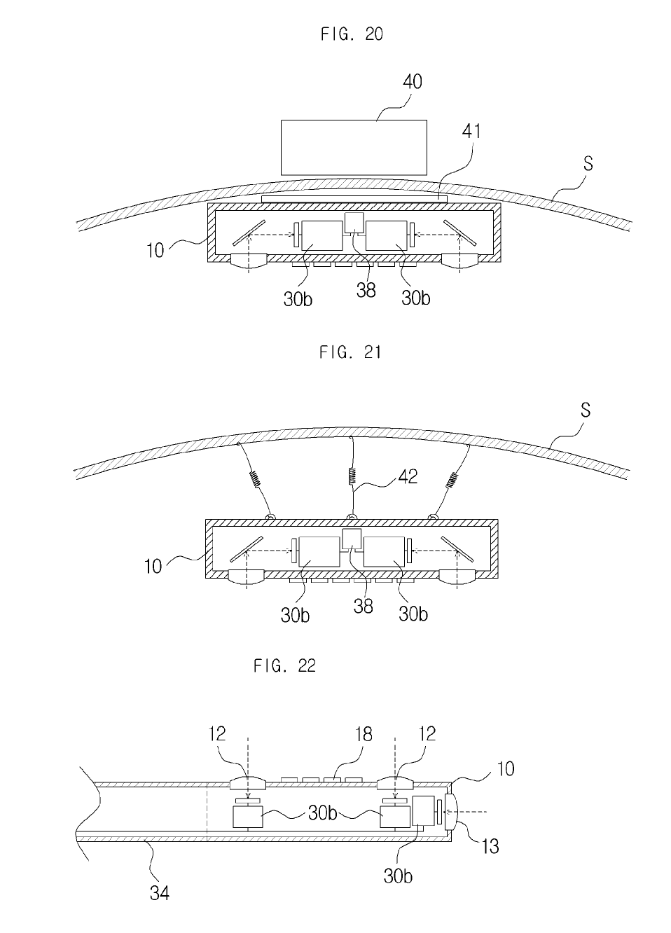

FIG. 11 through FIG. 13 are cross-sectional views of laparoscopes according to embodiments of the present invention, and FIG. 20 and FIG. 21 illustrate the use of certain laparoscopes according to embodiments of the present invention. Illustrated in FIG. 11 to FIG. 21 are a housing 10, lenses 12, a lighting part 18, image sensors 30b, an electrical contact 32, a support 34, and a needle 36.

In this embodiment, the image sensors 30b, such as CCD's, etc., may be built in directly inside the housing 10, so that the optical passage 20 of the previously described embodiment may be omitted. That is, a laparoscope according to the present embodiment can include a pair of lenses 12, which are set in the housing 10 with a particular distance in-between, and image sensors 30b, which receive image information from the lenses 12 and, without relaying the information to an optical passage 20, convert the information directly into electrical signals for transmission.

In this case, the image information converted into electrical signals can be transmitted by way of cables, etc., instead of the optical passage 20, so that the diameter of the laparoscope may be radically reduced to a level incomparable to conventional laparoscopes.

In the present embodiment, the image sensors 30b may be mounted within the housing 10 adjacent to the lenses 12, so that the light from the lenses 12 may be received directly by the image sensors 30b or may be altered by mirrors and then received by the image sensors 30b. The image sensors 30b can include a pair of CCD's corresponding with the lenses 12, respectively, to receive the light from the pair of lenses 12 and convert the light into electrical signals.

While FIG. 11 to FIG. 13 illustrate examples in which the light from the lenses 12 is reflected by mirrors to be received by the image sensors 30b, the present invention is not thus limited. It is obvious that the image sensors 30b can be set without mirrors directly behind the lenses 12 and that various other optical systems can be used for transferring the light from the lenses 12 to the image sensors 30b.

In this way, the light from the lenses 12 can be transferred to the image sensors 30b and converted into electrical signals, which in turn can be transmitted to the exterior through an electrical contact 32 connected to the image sensors 30b, so that the image photographed by the laparoscope may be displayed on a monitor. Thus, the image signals converted into electrical signals can be displayed on a monitor, allowing the surgeon to conduct surgery while viewing the image of the abdominal cavity on a screen.

As illustrated in FIG. 11, the housing 10, which may have an electrical contact 32 exposed on its exterior, may be inserted into the body, after which a cable (“C” in FIG. 11), etc., coupled to a support or a prop may be inserted in the body and electrically connected to the electrical contact

Also, as illustrated in FIG. 12, the housing 10 may be inserted into the body with a needle 36 coupled to the housing 10. Afterwards, the needle 36 may be protruded out of the body, and a cable, etc., may be connected to an electrical contact 32 formed on the needle 36. Thus, by having the electrical contact 32 protrude outwards through a needle 36, the electrical contact 32 can be exposed to the exterior simply by protruding the needle 36 out of the body.

Furthermore, by forming the electrical contact 32 at an end portion of the needle 36, as illustrated in FIG. 12, the laparoscope can be electrically connected with an external device by connecting a cable to the needle 36 protruding out of the body. By connecting the electrical contact 32 exposed through the needle 36 to an external device, the image information obtained within the abdominal cavity and converted into electrical signals can be displayed on an external monitor, etc.

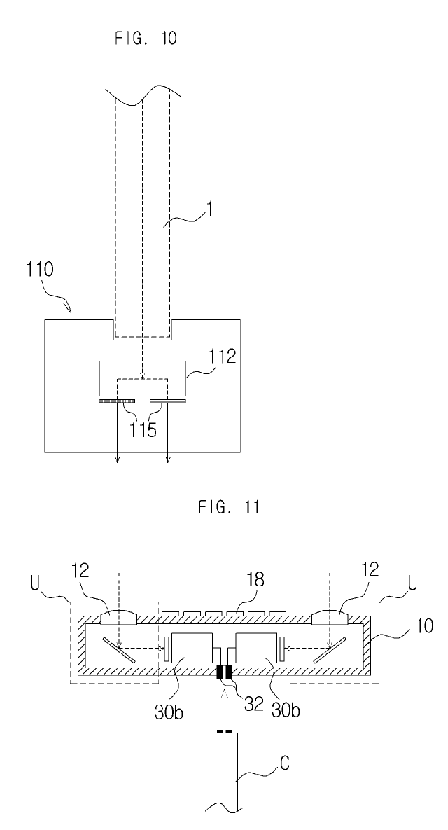

It is not necessary to have just one needle coupled to the housing 10, and as illustrated in FIG. 13, a multiple number of needles 36 may be coupled on, so that after the housing 10 is inserted into the body, a multiple number of needles 36 may be protruded out of the body. By thus coupling on a multiple number of needles 36, each needle can be made to serve as a support, so that the housing 10 may be prevented from rotating to an arbitrary orientation after it is inserted into the body. In addition, an electrical contact 32 can be formed on each of the needles 36, in case several electrical contacts are needed.

The housing 10 in this embodiment can be inserted into the measurement site for laparoscopic photography, and also can be referred to as the “laparoscope module.”

Of course, operating the laparoscope module 10 according to the present embodiment does not necessary require exposing an electrical contact at the exterior of the housing. As illustrated in FIG. 20 and FIG. 21, a transmitter can be connected to the image sensors 30b, so that the image information obtained by the laparoscope may be received on the outside by wired or wireless communication.

The transmitter according to this embodiment may serve to output or transmit the image information generated by the image sensor to an external device, and can be implemented in the form of an electrical contact 32 as described above or in the form of a wired communication device. As illustrated in FIG. 20 and FIG. 21, the transmitter can also be implemented as a wireless communication module 38 that exchanges data by wireless communication. Of course, various other types of devices can be used that are capable of providing the generated image information to an external device.

As illustrated in FIG. 20, a magnet 41 may be attached to the laparoscope module 10, and a different magnet 40 may be placed near the patient's skin (“S” in FIG. 20) from outside the body, so that the laparoscope module 10 inserted in the body may be moved to a desired position or secured at a particular position. For this purpose, the magnet 41 attached to the laparoscope module 10 and the magnet 40 manipulated outside the body can have poles that attract each other formed on opposing sides, such as an N-pole and an S-pole, or an S-pole and an N-pole, respectively.

Alternatively, as illustrated in FIG. 21, a hook 42 can be used to secure the laparoscope module 10 at a particular position after it is inserted into the body, with one end of the hook 42 connected to the laparoscope module 10 and the other end of the hook 42 suspended on the abdominal wall (“S in FIG. 21). In order to prevent the laparoscope module 10 suspended on the abdominal wall from being rotated arbitrarily, several hooks 42 can be used, as illustrated in FIG. 21, to secure the laparoscope module 10.

In order to receive the photographed image information after setting the laparoscope module 10 at a desired position within the body as described above, a power source (not shown), such as a battery, etc., can be built into the laparoscope module 10, for operating the image sensor 30b and the wireless communication module 38.

Furthermore, the laparoscope module 10 according to this embodiment does not necessarily have to include a built-in battery, and instead may be operated by power remotely supplied from an external power source, through the application of remote power supply techniques or communication techniques. For instance, induced current may be generated, by electromagnetic induction, from a relationship with a power supply device located outside the housing.

With the laparoscope module described above, the binocular disparity can be adjusted by adjusting the gap between the pair of lenses 12. That is, in a laparoscope module structure such as that illustrated in FIG. 11, a slide structure, etc., can be applied to the lens units (“U” in FIG. 11) on either side of the housing, so that the gap between the pair of lenses 12 may be adjusted.

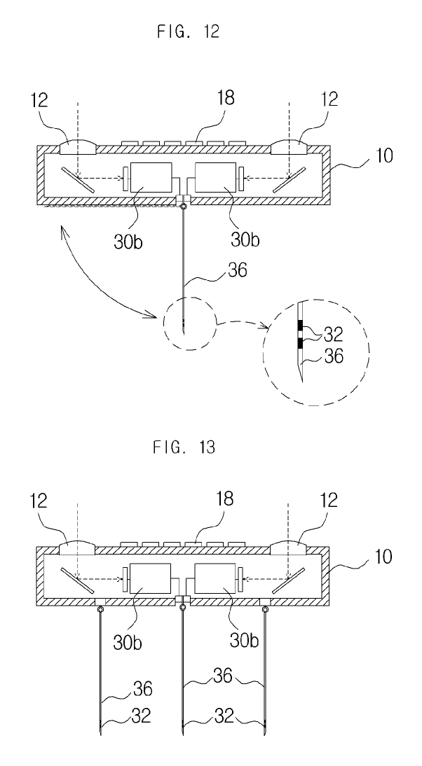

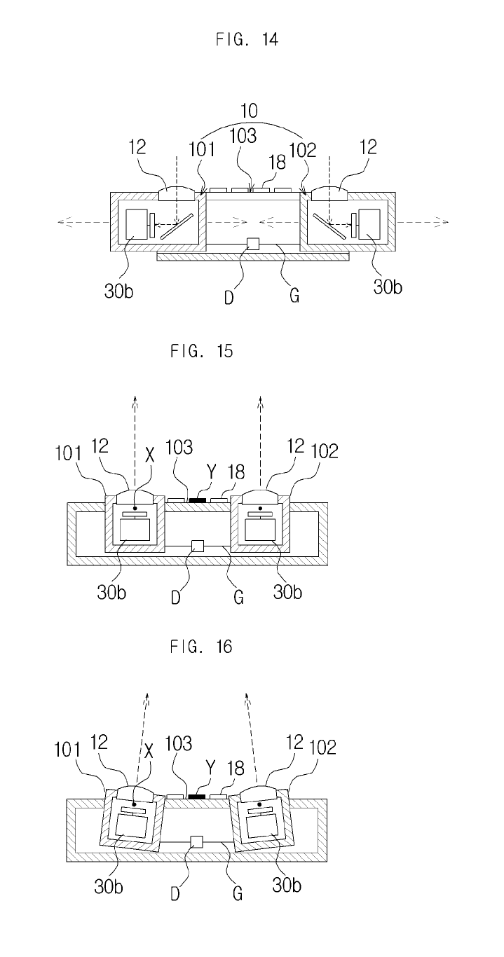

As illustrated in FIG. 14, the housing 10 can include a base unit 103 and module units 101, 102. The lenses 12 and the image sensors 30b can be held inside the module units 101, 102 to form lens modules, which may be protracted from and retracted in the base unit 103, whereby the gap between the pair of lenses 12 may be adjusted. In the example illustrated in FIG. 14, the pair of lenses 12 can be brought closer together almost until the lenses touch each other, and conversely can be separated apart from each other by the length of the housing 10.

Moving the lens modules, i.e. controlling the protraction and retraction of the module units 101, 102 with respect to the base unit 103, can be performed manually using preset positions, or can be controlled electronically by way of a driving unit such as a miniature motor (“D” in FIG. 14) built into the housing 10. A mechanical control method using a system of power-transferring means, such as wires and gears (“G” in FIG. 14), etc., may be used together with or separately from the above methods.

While FIG. 14 illustrates an example in which the module units 101, 102 located on both sides of the base unit 103 are configured to simultaneously move closer together or further apart, the invention is not thus limited. For example, it is also conceivable to have one end fixed to the housing and only the other end modularized as a unit that can be retracted and protracted with respect to the housing.

Moreover, in addition to adjusting the distance between the lens modules, the angle by which each module faces an object can be adjusted, just as the eyes of a person. For example, a lens module can be connected to two motors: one motor for moving the lens module left and right, and the other motor for moving the lens module up and down; so that the angle by which the lens module faces the object can be adjusted.

FIG. 15 and FIG. 16 illustrate a structure in which a miniature motor (D), etc., is used to rotate the lens modules 101, 102 on both sides in linkage with each other. That is, each lens module 101, 102 may be coupled to the housing such that the lens module 101, 102 is able to rotate about a particular axis (“X” in FIG. 15), while a driving unit, such as a motor (D), etc., and a power-transferring means, such as gears and wires (G), etc., which are linked to provide movement in opposite directions, may be coupled to each of the lens modules 101, 102, so that the two lens modules 101, 102 may be controlled to rotate simultaneously, as illustrated in FIG. 16. Of course, the means for rotating the lens modules do not necessarily have to include gears or wires, and various mechanisms may be applied, such as a four-bar linkage and a scissors-type linkage, etc.

In order to control the rotation angle of the lens modules 101, 102, a distance sensor, such as an ultrasonic sensor (“Y” in FIG. 15), etc., can be set between the two lenses. After the distance sensor measures the distance to an object, a “convergence angle” can be calculated, which is the angle at which the pair of lens modules may converge to obtain an image of the object, and the pair of lens modules can be controlled to rotate according to the calculated angle. In this way, the convergence of the pair of lens modules can be adjusted in the direction in which the sensor is set for measuring the distance.

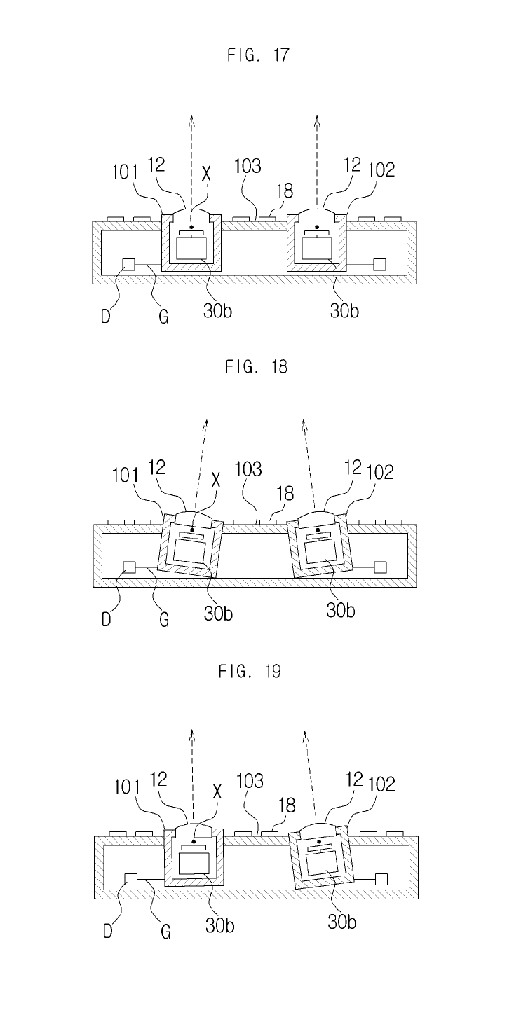

FIG. 17 through FIG. 19 illustrate a structure in which a driving unit, such as a miniature motor (D), etc., is coupled to each of the pair of lens modules, so that the lens modules 101, 102 on both sides can be rotated separately. That is, each lens module 101, 102 can be coupled to the housing such that the lens module 101, 102 is able to rotate about a particular axis (“X” in FIG. 17), and a driving unit, such as a motor (D) and a power-transferring means (G), can be coupled to each of the lens modules 101, 102. Then, not only may the pair of lens modules 101, 102 be rotated simultaneously for convergence, as illustrated in FIG. 18, but also each lens module 101, 102 may be rotated individually, as illustrated in FIG. 19.

By rotating the pair of lens modules in this manner, either in linkage or individually, it is possible to adjust the binocular disparity, just as the eyes of a person do.

In certain examples, the method of adjusting the rotation angles of the lens modules 101, 102 can also include analyzing the images obtained from the respective lenses for automatic focusing, and then adjusting the convergence angles accordingly.

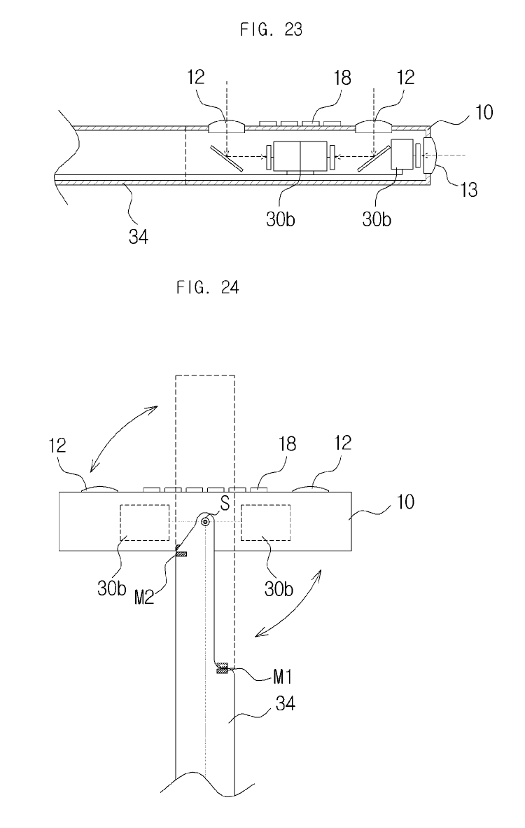

FIG. 22 and FIG. 23 are cross-sectional views of laparoscopes according to embodiments of the present invention, and FIG. 24 is a plan view of a laparoscope according to an embodiment of the present invention. Illustrated in FIG. 22 to FIG. 24 are a housing 10, lenses 12, a lighting part 18, optical receivers 30b, and a support 34.

According to the present embodiment, the housing 10 can be coupled with a support 34 that extends along a particular lengthwise direction, as illustrated in FIG. 22 to FIG. 24, instead of the optical passage 20 of the previously described embodiment. The support 34 may serve to provide mechanical support when the housing 10 is inserted into the abdominal cavity and may be detachably coupled with the housing 10.

A built-in cable in the support 34 can be electrically connected with the optical receivers 30b mounted in the housing 10. Then, the housing 10 may be inserted into the body while coupled to the support 34, and the electrical contact exposed at the other end of the support 34 may be connected with an external device.

As illustrated in FIG. 22 and FIG. 23, the support 34 can be coupled to the housing 10 such that the lengthwise direction of the support 34 matches the lengthwise direction of the housing 10. In this case, the laparoscope according to the present embodiment provides the advantage that the housing 10 can be inserted through the trocar and into the body directly while coupled to the support 34. In order to check the forward image during the process of inserting the laparoscope into the body, a separate auxiliary lens 13 can additionally be set into an end portion of the housing 10.

As illustrated in FIG. 24, the housing 10 can be hinge-coupled to the support 34, such that the housing 10 is able to rotate in relation to the support 34. When the laparoscope according to the present embodiment is inserted into the body, the housing 10 can be positioned to have the same lengthwise direction as that of the support 34, and after the laparoscope is inserted into the body, the housing 10 can be rotated such that the housing 10 is orthogonal to the support 34, to continue with the laparoscopic photography. In this case also, the housing 10 can be inserted into the body through the trocar while joined to the support 34, and afterwards the housing 10 can be rotated for use.

In order that the housing 10 may maintain its orientation after it has been rotated to have the same lengthwise direction as that of the support 34, a stopper (“S” in FIG. 24) can be formed near the hinge as illustrated in FIG. 25, or a pair of magnets (“M1” in FIG. 24) can be set in the appropriate positions of the housing 10 and the support 34 to serve as a stopper. It is also possible to form a stopper (“S” in FIG. 24, refer to FIG. 25) near the hinge or set a pair of magnets (“M2” in FIG. 24) in the appropriate positions of the housing 10 and the support 34 to serve as a stopper, for the purpose of maintaining the orientation of the housing 10 after it has been rotated to be orthogonal to the support 34.

While the above descriptions were provided using as an example a laparoscope inserted into the body of a patient, the laparoscope according to the present embodiment is not necessarily limited to inserting into the body for surgery. It is obvious that the laparoscope may be used for various other applications that include photographing the inside of an object that is difficult to observe with the naked eye, such as for observing a narrow space or the inside of a sealed container.

While the present invention has been described with reference to particular embodiments, it is to be appreciated that various changes and modifications can be made by those skilled in the art without departing from the spirit and scope of the present invention as defined by the appended claims.

1. A laparoscope comprising:
a housing extending a particular length;
a pair of lenses set in both end portions of the housing along a lengthwise direction;
a pair of first reflectors mounted within the housing adjacent to the pair of lenses, the first reflectors configured to reflect light from the pair of lenses towards a particular position;
a second reflector mounted within the housing, the second reflector configured to receive the light reflected from the pair of first reflectors and reflect the light in a particular direction;
an optical passage coupled to the housing, the optical passage configured to receive the light reflected from the second reflector and transmit the light to a particular position; and
a pair of first polarizing filters configured to polarize in different directions the light received through the pair of lenses respectively,
wherein the laparoscope includes a pair of the second reflectors corresponding respectively to the pair of first reflectors, and
wherein one of the pair of second reflectors comprises a two-way mirror to reflect light reflected from the corresponding first reflector and transmit light reflected by the other of the pair of second reflectors.

2. (canceled)

3. The laparoscope of claim 1, further comprising:
an optical splitter configured to receive the light transmitted by the optical passage and separate the light into two channels; and
a pair of second polarizing filters configured to respectively polarize the separated light in same directions as the pair of first polarizing filters.

4. A laparoscope comprising:
a housing extending a particular length;
a pair of lenses set in both end portions of the housing along a lengthwise direction;
a pair of first reflectors mounted within the housing adjacent to the pair of lenses, the first reflectors configured to reflect light from the pair of lenses towards a particular position;
a second reflector mounted within the housing, the second reflector configured to receive the light reflected from the pair of first reflectors and reflect the light in a particular direction; and
an optical passage coupled to the housing, the optical passage configured to receive the light reflected from the second reflector and transmit the light to a particular position,
wherein the optical passage is coupled to the housing along the same lengthwise direction as that of the housing, and
an auxiliary lens is set in an end portion along the lengthwise direction of the housing, the auxiliary lens for checking a forward image while the laparoscope is inserted into a body.

5. The laparoscope of claim 4, wherein a shutter is set in the housing, the shutter configured to selectively block the pair of lenses and the auxiliary lens.

6. A laparoscope comprising:
a housing extending a particular length;
a pair of lenses set in both end portions of the housing along a lengthwise direction;
an image sensor mounted within the housing, the image sensor configured to receive light through each of the pair of lenses, convert the light into electrical signals, and generate image information corresponding to the converted electric signals; and
a transmitter configured to transmit the image information to a receiver device.

7. The laparoscope of claim 6, further comprising:
a support detachably coupled to the housing, the support extending along a particular lengthwise direction,
wherein the support is coupled to the housing along the same lengthwise direction as that of the housing, and
wherein an auxiliary lens is set in an end portion along the lengthwise direction of the housing, the auxiliary lens for checking a forward image while the laparoscope is inserted into a body.

8. The laparoscope of claim 7, wherein a shutter is set in the housing, the shutter configured to selectively block the pair of lenses and the auxiliary lens.

9. (canceled)

10. The laparoscope of claim 6, further comprising:
a magnet coupled to the housing,
wherein the laparoscope, after being inserted into a body, is secured to a particular position by a magnetic force applied from out of the body.

11. The laparoscope of claim 6, wherein the laparoscope, after being inserted into a body, is secured to a particular position by a hook having one end thereof coupled to the housing and the other end thereof coupled to an abdominal wall of a patient.

12. (canceled)

13. (canceled)

14. The laparoscope of claim 6, wherein the housing comprises a base unit and a module unit, the module unit coupled to the base unit such that the module unit is retractable and protractible from the base unit, and
wherein the lenses and the image sensor are held in the module unit.

15. The laparoscope of claim 14, wherein a pair of module units are coupled respectively to both sides of the base unit, and the lens and the image sensor are held in each of the pair of module units such that retracting or protracting the pair of module units from the base unit decreases or increases a gap between the pair of lenses.

16. The laparoscope of claim 15, wherein the module unit is coupled to the base unit such that the module unit is rotatable about a particular axis, and rotating the module unit changes an angle by which the lens views an object.

17. The laparoscope of claim 16, wherein a distance sensor is coupled to the housing, the distance sensor configured to generate a sensing signal corresponding to a distance to the object, and
wherein the laparoscope further comprises:
a computing unit configured to receive a signal from the distance sensor and calculate a convergence angle of the pair of module units with respect to the object; and
a driving unit configured to rotate the module units in accordance with the angle calculated by the computing unit.

18. The laparoscope of claim 17, wherein the driving unit is coupled to the pair of module units such that the pair of module units are rotated in linkage.

19. The laparoscope of claim 17, wherein a pair of driving units are coupled respectively to the pair of module units such that the pair of module units are rotated separately.

20. The laparoscope of claim 16, further comprising:
a computing unit configured to analyze an image obtained from the image sensor, produce data corresponding to a focal length for the object, and calculate a convergence angle of the pair of module units from the produced data; and
a driving unit configured to rotate the module units in accordance with the angle calculated by the computing unit.

21. The laparoscope of claim 20, wherein the driving unit is coupled to the pair of module units such that the pair of module units are rotated in linkage.

22. The laparoscope of claim 20, wherein a pair of driving units are coupled respectively to the pair of module units such that the pair of module units are rotated separately.

23. A stereo adapter for mounting on a laparoscope to obtain a stereo image, the stereo adapter comprising:
an optical passage having the laparoscope inserted therein;
a housing rotatably coupled to the optical passage, the housing extending a particular length;
a pair of lenses set in both end portions of the housing along a lengthwise direction;
a pair of first reflectors mounted within the housing adjacent to the pair of lenses, the first reflectors configured to reflect light from the pair of lenses towards a particular position; and
a second reflector mounted within the housing, the second reflector configured to receive the light reflected from the pair of first reflectors and reflect the light towards the laparoscope.

24. (canceled)

25. (canceled)

26. (canceled)

27. (canceled)

28. (canceled)

29. The stereo adapter of claim 23, wherein the optical passage is coupled to the housing along the same lengthwise direction as that of the housing.

30. The stereo adapter of claim 23, wherein the housing is hinge-coupled to the optical passage, such that the housing is rotatable between an orientation having a same lengthwise direction as that of the optical passage and an orientation orthogonal to the optical passage.

31. A stereo adapter for mounting on a laparoscope, the laparoscope including a pair of first polarizing filters to polarize in different directions light received through a pair of lenses respectively and transmitting the polarized light to a single channel, the stereo adapter comprising:
an optical splitter configured to receive the light transmitted by the laparoscope and separate the light into two channels; and
a pair of second polarizing filters configured to respectively polarize the separated light in same directions as the pair of first polarizing filters.
